(19) United States
(12) Patent Application Publication
(10) Pub. No.: US 2023/0201134 A1
(43) Pub. Date: Jun. 29, 2023

(54) COMPOSITIONS FOR THE MANAGEMENT OF DEMYELINATING DISORDERS

(71) Applicants: Muhammed Majeed, Bangalore (IN); Kalyanam Nagabhushanam, East Windsor, NJ (US); Sarang Bani, Bangalore (IN); Anjali Pandey, Bangalore (IN)

(72) Inventors: Muhammed Majeed, Bangalore (IN); Kalyanam Nagabhushanam, East Windsor, NJ (US); Sarang Bani, Bangalore (IN); Anjali Pandey, Bangalore (IN)

(21) Appl. No.: 17/564,490

(22) Filed: Dec. 29, 2021

(51) Int. Cl. A61K 31/12; A61K 31/585; A61P 25/00

(52) U.S. Cl. CPC A61K 31/12; A61K 31/585; A61P 25/00

(57) ABSTRACT

The invention discloses a composition and method for therapeutically managing a demyelinated disorder through remyelination. The invention includes a composition comprising Withaferin A and Bisdemethoxycurcumin (BDMC), and a pharmaceutically or nutraceutically acceptable excipients, which can be effectively formulated as tablets and capsules. The invention also covers use of composition for effectively managing a demyelinated disorder through remyelination.

### CROSS REFERENCE TO RELATED APPLICATIONS

This is a non-provisional application claiming priority from Indian application no. 202141051709 filed on 11 Nov. 2021, the contents of which are incorporated herein by reference.

### FIELD OF INVENTION

The invention in general relates to compositions, use of compositions, and method of treating demyelinated disorders through remyelination. Specifically, the invention discloses reversal of demyelination in a mammal. Still more specifically, the invention discloses the remyelinating potential of a composition comprising bisdemethoxycurcumin (BDMC) and Withaferin A.

### BACKGROUND OF INVENTION

The term demyelination refers to the loss of the myelin sheath that surrounds and protects axons in the central nervous system. Demyelination occurs as a result of diseases that damage the myelin sheath or the cells that form it. The process of demyelination disrupts the electrical nerve conduction, which leads to symptoms of neurodegeneration. Based on the pathogenesis, demyelinating diseases are classified into several categories, demyelination due to inflammation, viral demyelination, demyelination by metabolic derangements, and demyelination by focal compression (Love, S, J. Demyelinating Diseases, Clin. Pathol. 2006 59(11), 1151-1159). Multiple sclerosis (MS), Balo's disease, Acute-Disseminated Encephalomyelitis (ADEM), Charcot-Marie-Tooth disease, Guillain-Barre Syndrome (GBS) HTLV-1 Associated Myelopathy (HAM), Neuromyelitis Optica (Devic's Disease), Schilder's disease, and Transverse Myelitis are diseases due to demyelination by inflammation and MS is the most common amongst all. MS is a neurodegenerative disease affecting the CNS, characterized by demyelination of axons or neuron loss, which is more prevalent and affects 50-300 cases per 100 000 individuals (Thompson A J, Baranzini S E, Geurts J, Hemmer b, Ciccarelli O. Multiple Sclerosis, Lancet 2018; 391; 1622-36). It is widely believed that the disease progression of M S in CNS is driven by adaptive immune response, consisting of T cells and B cells. Dendrou and coworkers had reviewed the role of innate immune response in the initiation and progression of M S (Dendrou C A, Fugger L, Friese M A, Immunopathology of multiple sclerosis. Nat. Rev. Immunol. 2015; 15; 545-548). A rare variant of MS is Balo's disease. It is a rare demyelinating disorder of the central nervous system (CNS) in which the myelin is damaged. ADEM's disease is another inflammatory disease affecting the brain, spinal cord and damages the myelin, especially in children. Charcot-Marie-Tooth disease causes damage to the peripheral nerves, affecting signal transmission from brain and spinal cord to rest of the body and affects the nerves controlling the muscles. Guillain-Barre Syndrome (GBS), an autoimmune disease wherein the immune system attacks healthy nerve cells of the peripheral nervous system (PNS). This disease is usually triggered by gastroenteritis or a lung infection. HAM is a slow progressive, chronic disease of the spinal cord seen in some people infected with the HTLV-1 virus, which results in painful stiffness and weakness of the legs. Neuromyelitis Optica (Devic's Disease) is a rare condition where the immune system damages the spinal cord and optic nerves. Schilder's disease inflammatory demyelinating disorder of the central nervous system characterized by demyelination of vast areas of the white matter. Transverse Myelitis is a neurological condition resulting in inflammation of the spinal cord.

The membrane composition of myelin is 70% lipids and 30% proteins, wrapped around axons forming a sheath. In the CNS, oligodendrocytes. especially oligodendrocyte precursor cells (OPCs) and mature oligodendrocytes, are responsible for the synthesis of the membrane and remyelination, Oligodendrocytes also plays a major role in axonal functional integrity, in addition to playing a role in conduction, it also provides lactate as an energy source for axonal transport (Catherine Lubetzki, Bernard Zalc, Anna Williams, Christine Stadelmann, Bruno Stankof; Remyelination in multiple sclerosis: from basic science to clinical translation 2020; 19; 678-688). The nerve impulses are more evenly distributed in non-myleinated fibres than in myelinated ones, this uneven distribution is attributed to the sodium channels clustered at the nodes of Ranvier, resulting in faster transmission (at least 50 to 100 times) of action potential in myelinated axons.

Demyelination results from inability to make and maintain myelin sheath by oligodendrocyte. This primary demyelination can arise from genetic abnormalities and in the case of multiple sclerosis, inflammatory damage to myelin and oligodendrocyte, eventually leading to loss of myelin internode and thereby conduction block (Robin J. M. Franklin, Charles Ffrench-Constant, Remyelination in the CNS: from biology to therapy, 2008; 9; 839-855). There have been numerous causes attributed to demyelination such as causative inflammation, auto-immune process, metabolic, damage to blood vessel in brain, loss of oxygen, physical compression and viral infection. The symptoms associated with demyelinating disorders include vision loss, lack of neuromuscular coordination, muscle weakness, non-rhythmic heartbeat or palpitations, loss of bladder/bowel control, sensory changes, numbness, loss of reflexes, poorly controlled blood pressure, dizziness, memory problems, pain, and fatigue,

The process of restoring the entire myelin sheath to the demyelinated axons is known as the remyelination or myelin repair, which restores saltatory conduction (Smith K. J, Blakemore, W. F, and McDonald W. I, Central Remyelination Restores Secure Conduction, Nature 1979; 280; 395-396). Experimental evidence has shown that transgenic mice lacking genes encoding myelin sheath protein exhibited long term axon degeneration, and it is proven that remyelination resulting in even thin myelin sheath are sufficient for axon survival. (Griffiths et al. Axonal Swellings and degeneration in mice lacking the major proteolipid of myelin, Science 1998; 280; 1610-1613; Kornek. B et al. Multiple Sclerosis and Chronic Autoimmune Encephalomyelitis: a comparative quantitative study of axonal injury in active, inactive, and remyelinated lesions, Am. J. Pathol. 2000; 157; 267-276). Further, evidence has been gathered that remyelination results in greater number of oligodendrocytes in the remyelinating area compared to those undergoing remyelination, and remyelination occurs only in the area depleted of oligodendrocytes (Prayoonwiwat. N, Rodriguez. M The Potential for oligodendrocyte proliferation during demyelinating disease, J. Neuropathol. Exp. Neurol. 1993; 52; 55-63; Sim, F. J, Zhao, C, Penderis, J, Franklin R. J. M, The age-related decrease in CNS remyelination efficiency is attributable to an impairment of both oligodendrocyte progenitor recruitment and differentiation, (J. Neurosci. 2002; 22; 2451-2459).

WO 2017060750 A1 discloses withania extract for the treatment of demyelinating diseases. This application relates to the use of a composition from a plant extract of Withania somnifera in combination with wide array of extracts from different plant source including Curcuma longa, for treating or limiting development of pathologies of the myelin, including multiple sclerosis disease. But the invention discloses use of the wholesome extracts for treating multiple sclerosis without pinning down on the actual ingredient or the pathway by which remyelination occurs.

WO 2004/075883 A1 and US 2004/0167217 A1 discloses a method of preventing or treating disease associated with cell death in neuronal cells, central and peripheral nervous system cells, which involves administration of polyphenolic compound (A) or its analogs, consisting of curcumin, analogs of curcumin, caffeic acid phenethyl ester, and analogs of caffeic acid phenethyl ester. But no experiments or data were shown to treat specifically on demyelinated cases.

Hwang et al. disclosed use of Withania Somnifera (Ashwagandha in Sanskrit) and two of its metabolites, withanolide A and withaferin A, for increased neuronal survival under nutrient deprived conditions. The authors were able to show neuroprotection mediated activation of Akt/PI-3K and MAPK cascades, CREB phosphorylation, and BDNF production. (Dahae Hwang, Isabel Vasquez, Leticia Galvez, Huong Do, Anthony Lopez de Santa Ana, Shane Matta, Feimeng Zhou, Michael Chen and Amelia Russo-Neustadt, Ashwagandha and Its Active Ingredient, Withanolide A, Increase Activation of the Phosphatidylinositol 3′ Kinase/Akt Cascade in Hippocampal Neurons. 2017; 20(2) 1-19).

US 2019/0328750 A1 discloses a method of promoting remyelination in demyelinated disorders using steroid hormone and a sonic hedgehog signaling pathway modulating agent.

Curcumin has been used in an oral composition in combination with others such as N-acetyl-L-carnitine, α-lipoic acid in the treatment of neuropathies (IL250594 A), but no experiments or data were shown to treat specifically on demyelinated cases.

IN201841016468 discloses herbal formulation useful for arresting inflammation and degeneration comprises curcumin, black pepper, Tinospora cordifolia, and Phyllanthus niruri, Phyllanthus amarus or Phyllanthus urinaria. Curcumin extract was used along with other extracts, and piperin was used merely to improve the bioavailability and absorption of curcuminoids.

Curcumin that is commercially available includes three curcuminoids, 72 to 77% curcumin, 14 to 18% dimethoxy curcumin, and 3 to 5% bis-demethoxy curcumin. And larger fraction of curcumin makes it hydrophobic and thereby affecting bioavailability and absorption (Pushpakumari, K. N, Vargheese, Kottol, K, Enhancing the Absorption of Curcuminoids from Formulated Turmeric Extracts, 2015; 6(6) 2468-2476). The biological properties of curcumin, bisdemethoxycurcumin and demethoxycurcumin vary in different diseases conditions and recently bisdemethoxycurcumin and demethoxycurcumin are garnering the much attention owing to their similar and superior efficacy over curcumin in managing certain disease conditions. (Majeed et al., Reductive Metabolites of Curcuminoids, Nutriscience Publishers LLC, 2019).

The treatment for demyelination disorders continues to evolve starting from the use of Interferon β-1a, Interferon β-1b, Glatiramer acetate, Dimethyl fumarate, Teriflunomide, Natalizumab, Alemtuzumab, and Ocrelizumab. Most of the drugs have either been associated with severe liver toxicity, suffer from multiple doses of administration or higher relapse rate. There is a constant need and search for effective treatment for Demyelinating disorders, especially for MS.

### Objectives of the Invention

It is a general objective of the invention to disclose a composition comprising Withaferin A and Bisdemethoxycurcumin (BDMC) to therapeutically manage demyelinating disorder in a mammal by promoting remyelination

It is the main objective of the invention, to disclose a composition comprising Withaferin A and Bisdemethoxycurcumin (BDMC), wherein Withaferin A and BDMC are in 3:1 w/w.

In another objective of the invention, to disclose a method for therapeutic management of demyelinating disorders in a mammal, comprising identifying a mammal showing symptoms of demyelination and administering to the mammal an effective dose of a composition comprising Bisdemethoxycurcumin (BDMC) and Withaferin A, to bring about the effect of promoting remyelination.

In yet another objective of the invention covers use of a composition comprising Bisdemethoxycurcumin (BDMC) for therapeutic management of a demyelinating disorder in a mammal.

In yet another objective of the invention is a method of promoting remyelination of a mammalian neuron, said method comprising: bringing into contact an effective dose of a composition comprising Bisdemethoxycurcumin (BDMC) to the said mammalian neuron, showing characteristic of demyelination, to bring about the effect of promoting remyelination.

In yet another objective of the invention covers use of a composition for promoting remyelination of a mammalian neuron comprising Bisdemethoxycurcumin (BDMC).

### SUMMARY

The invention broadly solves the aforementioned problems mentioned in the background by covering a composition comprising Withaferin A and Bisdemethoxycurcumin (BDMC), wherein Withaferin A and BDMC are in 3:1 w/w. The invention further covers a method, use of composition for treating demyelinating disorders in a mammal and a mammalian neuron comprising Withaferin A and at least 20% Bisdemethoxycurcumin (BDMC).

The first aspect of the invention relates to a composition comprising Withaferin A and Bisdemethoxycurcumin (BDMC).

The second aspect of the invention covers a method for therapeutic management of a demyelinating disorder in a mammal, said method comprising steps of:
(a) identifying a mammal showing symptoms of demyelination; and
(b) administering to the mammal, an effective dose of a composition comprising Bisdemethoxycurcumin (BDMC), to bring about the effect of promoting remyelination.

Yet another aspect of the invention covers use of a composition comprising Bisdemethoxycurcumin (BDMC) for therapeutic management of a demyelinating disorder in a mammal

In yet another aspect of the invention covers a method of promoting remyelination of a mammalian neuron, said method comprising: bringing into contact an effective dose of a composition comprising Bisdemethoxycurcumin (BDMC) to the said mammalian neuron, showing characteristic of demyelination, to bring about the effect of promoting remyelination.

In yet another aspect of the invention, covers Use of a composition comprising Bisdemethoxycurcumin (BDMC) for promoting remyelination of a mammalian neuron.

The broader scope of applicability of the present invention will be apparent from the detailed description below. However, it should be understood that the detailed description and specific examples, while indicating preferred embodiments of the invention, should not be construed as the limitations to the invention, and it is within the scope of those skilled in the art to make various changes and modifications, such as changing the concentration range of composition used, derivatives/analogs of Withaferin A and BDMC, experimental conditions, choice of mammals, to the spirit and scope of the invention from this detailed description.

### BRIEF DESCRIPTION OF DRAWINGS

The patent or application file contains at least one drawing executed in color. Copies of this patent or patent application publication with color drawing(s) will be provided by the office upon request and payment of the necessary fee.

FIG. 1 is a dose response curve of Withaferin A and BDMC, for measure of Myelin Oligodendrocyte Glycoprotein (MOG).

FIG. 2 is a dose response curve Withaferin A and BDMC, for measure of Brain Derived Neurotrophic Factor (BDNF).

FIG. 3 shows measure of proliferation of Glycoprotein biomarker, Myelin basic protein (MBP), in the presence of Withaferin A, BDMC and combination thereof. Values are expressed as Mean±S.E.Number of animals in each group: 10 p value: *<0.01; **<0.001

FIG. 4 shows upregulation of growth factors biomarker, Platelet Derived Growth Factor Receptor alpha (PDGFRa), Values are expressed as Mean±S.E. Number of animals in each group: 10, p value: *<0.01; **<0.001 and FIG. 5 shows upregulation of growth factors biomarker Chondroitin Sulfate Proteoglycan 4 (NG2), in the presence of Withaferin A, BDMC and combination thereof. Values are expressed as Mean±S. E. Number of animals in each group: 10, p value: *<0.01:

FIG. 6 shows downregulation of inflammatory biomarker, Transient receptor potential ankyrin 1 (TRPA1), (Values are expressed as Mean±S.E. Number of animals in each group: 10, p value: *<0.01)

FIG. 7 shows downregulation of inflammatory biomarker, Amphoterin-induced protein 3 (AMIGO3)(Values are expressed as Mean±S.E. Number of animals in each group: 10 p value: *<0.05: **<0.01; **<0.001), FIG. 8 shows downregulation of inflammatory biomarker, C-reactive Protein (CRP)(Values are expressed as Mean±S.E. Number of animals in each group: 10 p value: *<0.01: *<0.001), and FIG. 9 shows downregulation of inflammatory biomarker CXC chemokine receptor 2(CXCR2) in the presence of Withaferin A, BDMC and combination thereof (Values are expressed as Mean±S.E. Number of animals in each group: 10 p value: *<0.05; **<0.01; ***<0.001).

FIG. 10 shows effect of in Withaferin A, BDMC and combination thereof neuromuscular coordination, grip strength. (Values are expressed as Mean±S.E. Number of animals in each group: 10, p value: *<0.05.

Immunohistochemistry of cortical layer of Normal control (FIG. 11), demyelinated control (FIG. 12), in the presence of Withaferin A (FIG. 13), BDMC (FIG. 14) and the combinations thereof (FIG. 15).

### DESCRIPTION OF PREFERRED EMBODIMENTS

### Selected Definitions

All the terms used in this application carry ordinary meaning as known in the prior art unless otherwise specified. Few other specific definitions used in this invention are explained below, which applies throughout this specification. Claims provide broader definition unless and otherwise specified.

Withaferin A used in this invention can be isolated from Solanaceae family, Physalis longifolia, Vassobia breviflora, and Withania somnifera. Bisdemethoxycurcumin (BDMC) used in this invention can be isolated from Curcuma longa.

Therapeutically managing or management refers to a condition of effectively ameliorating disorders disclosed in the invention. An effective dose refers to positive or modulatory effects of remyelination in mammals covered under this invention.

The invention covers a composition comprising Withaferin A and Bisdemethoxycurcumin (BDMC), wherein Withaferin A and BDMC are in 3:1 w/w. The invention further covers a method, use of composition for treating demyelinating disorders in a mammal and a mammalian neuron comprising Withaferin A and at Bisdemethoxycurcumin (BDMC).

In the most preferred embodiment, the invention discloses a composition comprising Withaferin A and Bisdemethoxycurcumin (BDMC). In a related embodiment, wherein Withaferin A and BDMC are in 1:1 w/w, or more preferably 1:2 w/w, or more preferably 1:3 w/w, or more preferably 2:1 w/w, or most preferably 3:1 w/w.

In a related aspect of this and other embodiments of the invention, the composition further comprises stabilizing agents, bioavailability enhancers and antioxidants, pharmaceutically or nutraceutically or cosmeceutically accepted excipients and enhancers, administered orally in the form of tablets, capsules, syrups, gummies, powders, suspensions, emulsions, chewables, candies or eatables.

In another most preferred embodiment of the invention, the invention discloses a method for therapeutic management of a demyelinating disorder in a mammal, said method comprising steps of:
(a) identifying a mammal showing symptoms of demyelination; and
(b) administering to the mammal, an effective dose of a composition comprising Bisdemethoxycurcumin (BDMC), to bring about the effect of promoting remyelination.

In a related aspect of this and other embodiments of the invention, wherein the composition further comprises withaferin A. In a related aspect of this and other embodiments of the invention, the effective dose of withaferin A or BDMC is individually selected from 3 mg/kg to 55 mg/kg of bodyweight of a mammal, more preferably between 10 to 30 mg/kg, and most preferably between 10 to 25 mg/kg of the said mammal. In a related aspect of this and other embodiments of the invention the total effective dose of the composition is selected from 30 to 40 mg/kg bodyweight, and most preferably between 35 to 40 mg/kg bodyweight of the said mammal. In further aspect of this and other embodiments, wherein the dose of Withaferin A is 10 mg/kg and BDMC is 27.5 mg/kg in the total composition of the said mammal, or more preferably the dose of Withaferin A is 12.5 mg/kg and BDMC is 25 mg/kg in the total composition of the said mammal, or more preferably the dose of Withaferin A is 15 mg/kg and BDMC is 12.5 mg/kg in the total composition of the said mammal, or more preferably the dose of Withaferin A is 20 mg/kg and BDMC is 17.5 mg/kg in the total composition of the said mammal, or more preferably the dose of Withaferin A is 17.5 mg/kg and BDMC is 20 mg/kg in the total composition of the said mammal, or more preferably the dose of Withaferin A is 20 mg/kg and BDMC is 20 mg/kg in the total composition of the said mammal, or most preferably the dose of Withaferin A is 25 mg/kg and BDMC is 12.5 mg/kg in the total composition of the said mammal.

In a related aspect of this and other embodiments of the invention, Withaferin A was isolated from the source, using alcohol as the solvent and concentrating the extract after multiple extractions from the alcohol. Further, treating the concentrated extract with water, and extracting the aqueous layer with organic solvents, non-polar and polar, to yield Withaferin A.

In a related aspect of this and other embodiments of the invention, the total curcuminoids in the composition are in the range of not less than 20% w/w. In a further aspect, the composition is isolated and formulated using a process well known in scientific literature:

Further aspect of this and other embodiments of the invention, wherein demyelinating disorders are selected from the group consisting of multiple sclerosis, Acute Disseminated Encephalomyelitis (ADEM), Balo's Disease (Concentric Sclerosis), Charcot-Marie-Tooth Disease (CMT), Guillain-Barre Syndrome (GBS), HTLV-I Associated Myelopathy (HAM), Neuromyelitis Optica (Devic's Disease), Schilder's disease, and Transverse Myelitis. In further aspect of this and other embodiments of the invention, demyelinating disorder is preferably Acute Disseminated Encephalomyelitis (ADEM), or preferably Balo's Disease (Concentric Sclerosis), or preferably Charcot-Marie-Tooth Disease (CMT), or preferably Guillain-Barre Syndrome (GBS), or preferably HTLV-I Associated Myelopathy (HAM), or preferably Neuromyelitis Optica (Devic's Disease), or preferably Schilder's disease, or preferably Transverse Myelitis or most preferably Multiple Sclerosis (MS). In a related aspect of this and other embodiments of the invention, wherein the cause of demyelination is selected from the group consisting of anti-inflammatory, auto-immune, metabolic, damage to blood vessels in the brain, viral infections, and chemically induced. In a related aspect of this and other embodiments of the invention, the symptoms of demyelination disorders are selected from the group consisting of vision loss, lack of neuromuscular coordination, muscle weakness, non-rhythmic heartbeat or palpitations, loss of bladder and bowel control, sensory changes, numbness, loss of reflexes, poorly controlled blood pressure, dizziness, memory problems, pain, and fatigue.

In further aspect of this and other embodiments of the invention, wherein management of the demyelinating disorder in the mammal is brought about by promoting remyelination, by increasing proliferation of glycoproteins involved in myelin formation, upregulation of growth factors, down regulation of inflammatory markers and increasing neuromuscular coordination.

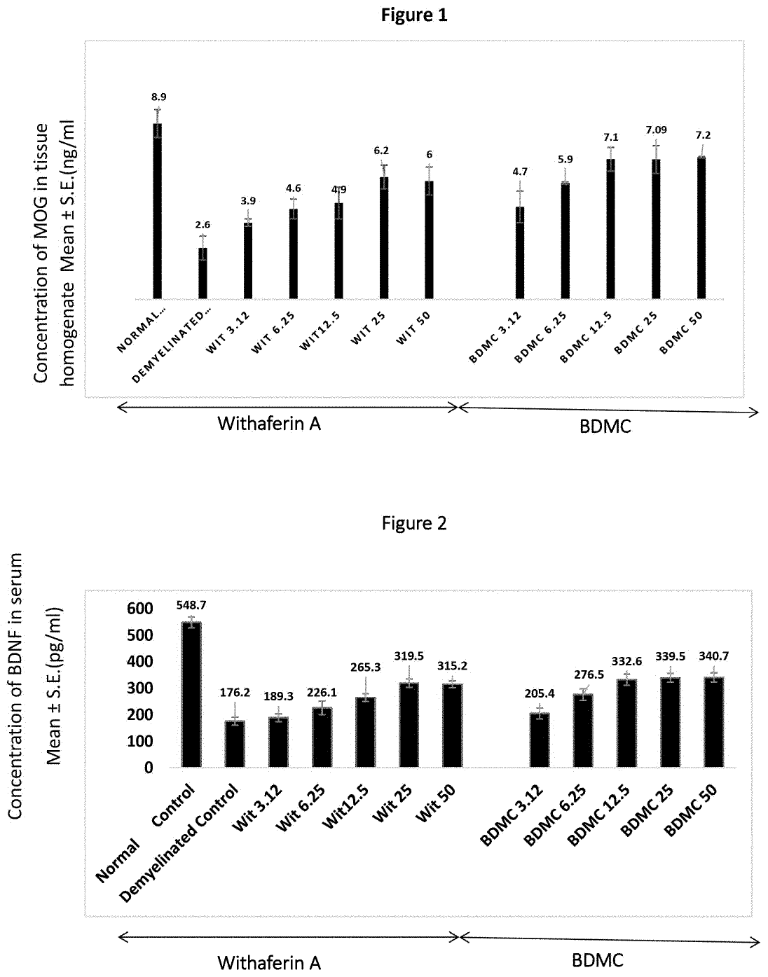

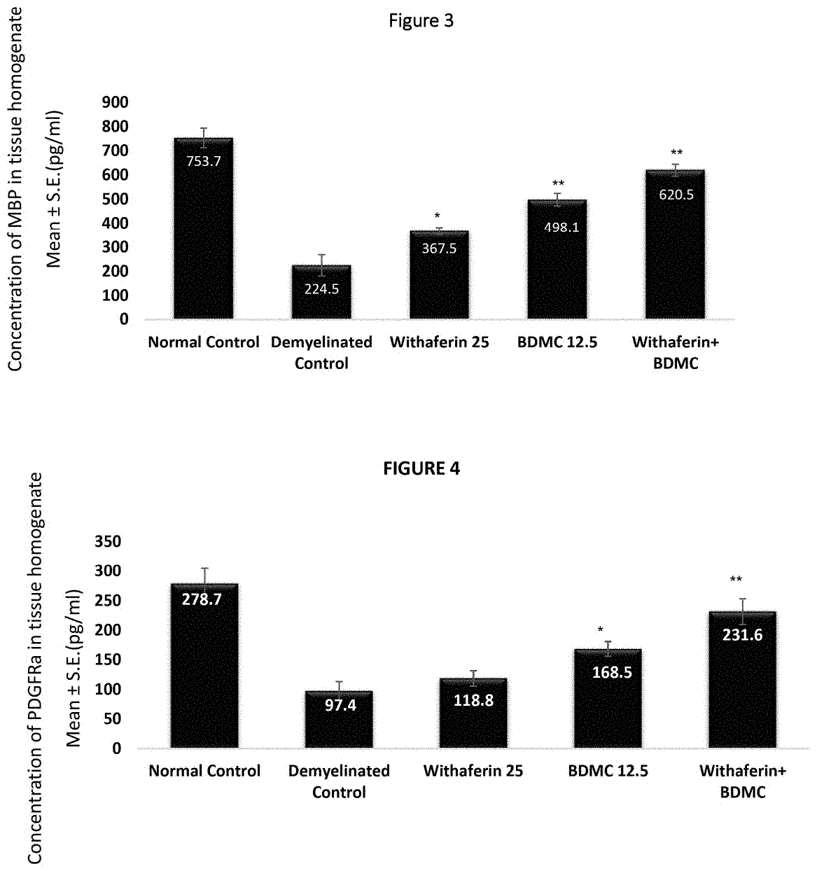

In related aspect to this and other related embodiments of the invention, wherein glycoproteins are selected from the group consisting of Myelin basic protein (MBP) and Myelin Oligodendrocyte Glycoprotein (MOG). Further, in this and other related aspect of this embodiment, Withaferin A or BDMC resulted in proliferation of MOG by at least 125%, preferably by 150%, or preferably by 175%, or more preferably by 188%, or most preferably by 225% (FIG. 1). Further, in this and other related aspect of this embodiment, Withaferin A or BDMC resulted in proliferation of Myelin basic protein (MBP) by at least 50%, or most preferably by 60%, or more preferably by 100%. The combination of Withaferin A and BDMC resulted in proliferation of MBP by at least 100%, or most preferably by 175%, compared to the demyelination control (FIG. 3).

In related aspect to this and other related embodiments of the invention, wherein the growth factors are selected from the group consisting of Platelet Derived Growth Factor Receptor alpha (PDGFRa) and Chondroitin Sulfate Proteoglycan 4 (NG2). Further, in this and other related aspect of this embodiment, Withaferin A or BDMC resulted in upregulation of PDGFRa by at least 20%, or by at least 30%, or more preferably by at least 50% or most preferably by 70%. The combination of Withaferin A and BDMC resulted in upregulation of PDGFRa by at least 50%, or more preferably by at least 100%, or most preferably by at least 125%, compared to the demyelination control (FIG. 4). Further, in this and other related embodiments of the invention, Withaferin A or BDMC resulted in upregulation of NG2 by at least by 30%, or more preferably by at least 50% or most preferably by 90%. The combination of Withaferin A and BDMC resulted in upregulation of NG2 by at least 50%, or more preferably by at least 125%, or most preferably by at least 150% (FIG. 5).

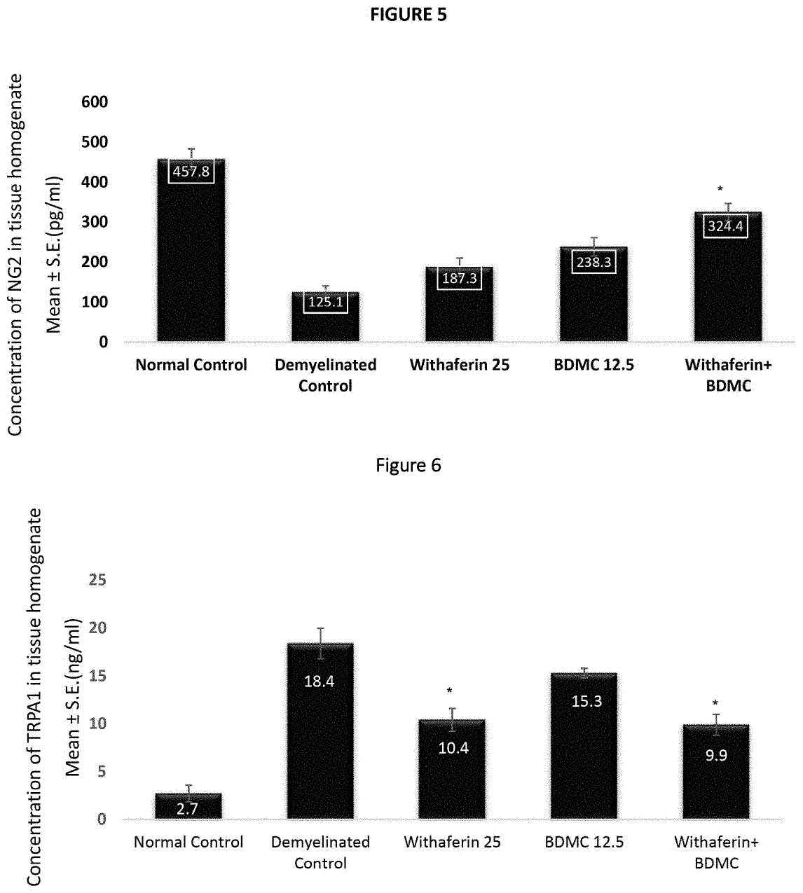

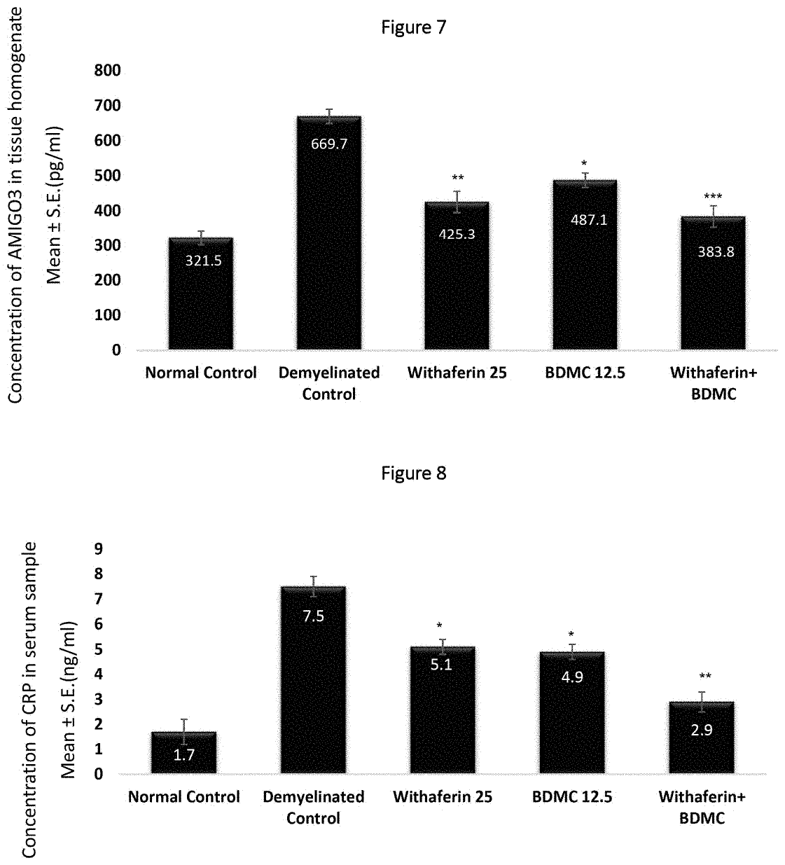

In related aspect to this and other related embodiments of the invention, wherein the inflammatory markers are selected from the group consisting of Transient receptor potential ankyrin 1 (TRPA1), Amphoterin-induced protein 3 (AMIGO3), C-reactive Protein (CRP), and CXC chemokine receptor 2 (CXCR2). Further in this and other related aspect of the embodiment of this invention, Withaferin A or BDMC resulted in down regulation of Transient receptor potential ankyrin 1 (TRPA1) inflammatory marker, by at least 15%, or by at least 20%, or more preferably between 40 to 50%. The combination of Withaferin A and BDMC resulted in down regulation of Transient receptor potential ankyrin 1 (TRPA1) inflammatory marker, by at least 25%, or by at least 30%, or most preferably by 45% (FIG. 6). Further in this and other related aspect of the embodiment of this invention, Withaferin A or BDMC resulted in down regulation of Amphoterin-induced protein 3 (AMIGO3) inflammatory marker by at least 25%, or by at least 30%, or more preferably by 35%. The combination of Withaferin A and BDMC resulted in down regulation of AMIGO3 inflammatory marker by at least 30%, and most preferably by 40% (FIG. 7). Further in this and other related aspect of the embodiment of this invention, Withaferin A or BDMC resulted in down regulation of C-reactive Protein (CRP) inflammatory marker by at least 30%, or most preferably by 35%. The combination of Withaferin A and BDMC resulted in down regulation of CRP inflammatory marker by at least 50%, or most preferably by 60% (FIG. 8). Further in this and other related aspect of the embodiment of this invention, Withaferin A or BDMC resulted in down regulation of CXC chemokine receptor 2 (CXCR2) inflammatory marker by at least 15%, or more preferably by 20%, or most preferably by at least 25%. The combination of Withaferin A and BDMC resulted in down regulation of CXCR2 inflammatory marker by at least 50%, or preferably by at least 55%, or most preferably by 65% (FIG. 9).

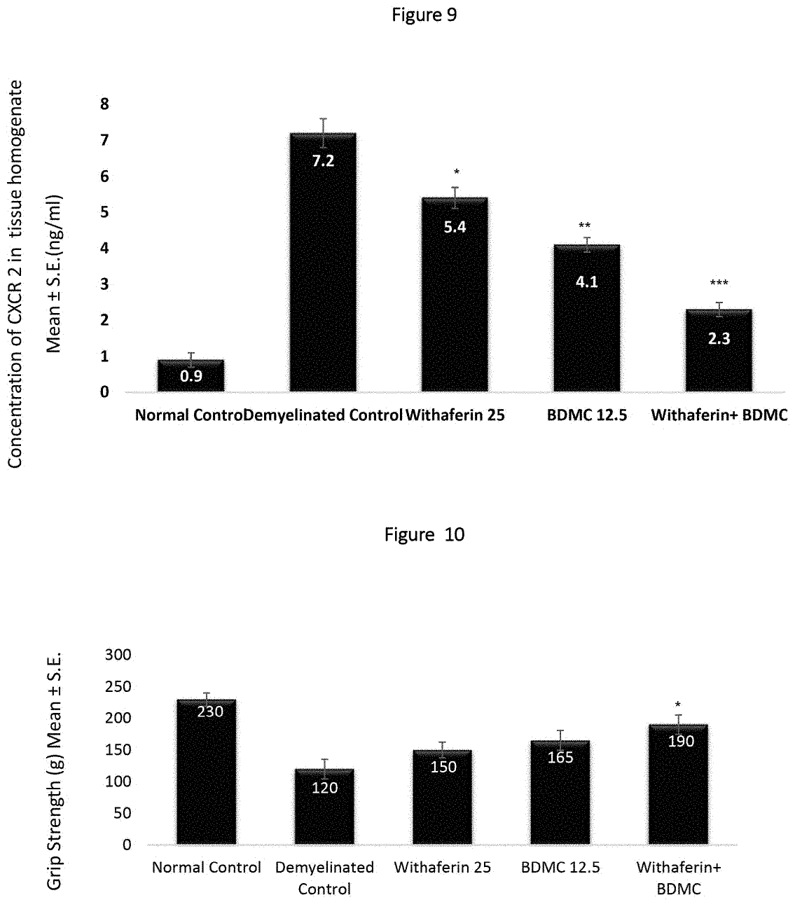

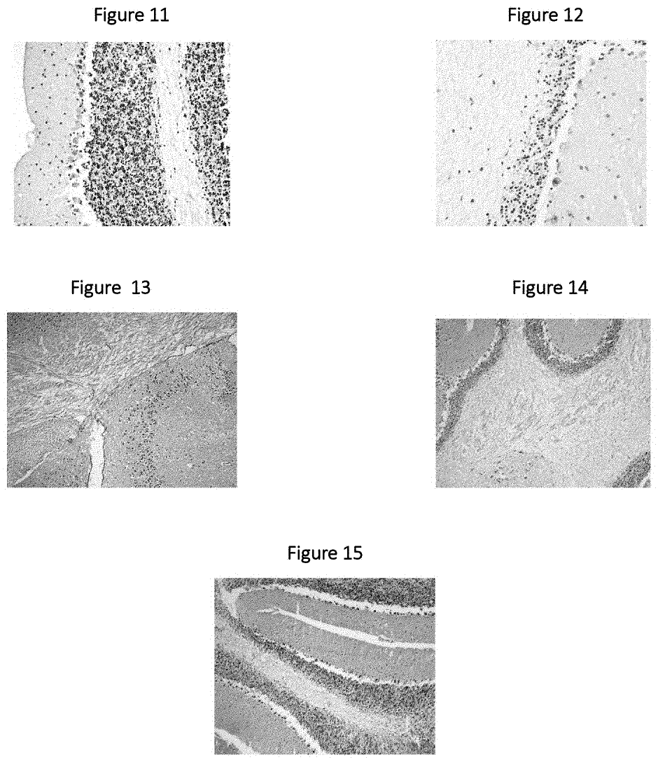

In related aspect to this and other related embodiments of the invention, wherein neuromuscular coordination is selected from the group consisting of grip strength, motor coordination, learning and locomotor activity. Further in this and other related aspect of the embodiment of this invention, Withaferin A or BDMC resulted in improved grip strength by at least 20%, or more preferably by at least 25%, or most preferably by 30%. The combination of Withaferin A and BDMC resulted in improved grip strength by at least 50%, or most preferably by 55% (FIG. 10). Further in this and other related aspect of the embodiment of this invention, Withaferin A or BDMC resulted in improved motor co-ordination in 5 out of 10 mammals, whereas the combination of Withaferin A and BDMC resulted in improved motor co-ordination in 7 out of 10 mammals, compared to 3 out of 10 in demyelinated control mammals. Further in this and other related aspect of the embodiment of this invention, Withaferin A showed 30 to 40% improvement in the spontaneous or induced or locomotor activity compared to the demyelinated control, BDMC showed 25 to 30% improvement, and the combination showed 60 to 65% improvement compared to the demyelinated control.

In another most preferred embodiment of the invention, a method for therapeutic management of a demyelinating disorder in a mammal, said method comprising steps of:
(a) identifying a mammal showing symptoms of demyelination; and
(b) administering to the mammal, an effective dose of a composition comprising Bisdemethoxycurcumin (BDMC), to bring about the effect of promoting remyelination.

In related aspect of this and other embodiments of the invention, wherein the composition further comprises withaferin A. The concentration range of doses, choice of mammals, selection of demyelination disorders, markers associated with promotion of remyelination, formulation for administration, explained in the previous embodiments are applicable to this embodiment

In yet another most preferred embodiment, the invention discloses Use of a composition comprising Bisdemethoxycurcumin (BDMC) for therapeutic management of a demyelinating disorder in a mammal.

In related aspect of this and other embodiments of the invention, wherein the composition further comprises withaferin A. The concentration range of doses, choice of mammals, selection of demyelination disorders, markers associated with promotion of remyelination, formulation for administration, explained in the previous embodiments are applicable to this embodiment

In yet another most preferred embodiment of the invention discloses a method of promoting remyelination of a mammalian neuron, said method comprising: bringing into contact an effective dose of a composition comprising Bisdemethoxycurcumin (BDMC) to the said mammalian neuron, showing characteristic of demyelination, to bring about the effect of promoting remyelination. In further aspect of this and other embodiments of the invention embodiment, wherein the composition further comprises withaferin A. The concentration range of doses, choice of mammals, selection of demyelination disorders, markers associated with promotion of remyelination, formulation for administration, explained in the previous embodiments are applicable to this embodiment

In yet another most preferred embodiment of the invention discloses use of a composition comprising Bisdemethoxycurcumin (BDMC) for promoting remyelination of a mammalian neuron. In further aspect of this and other embodiments of the invention embodiment, wherein the composition further comprises withaferin A. The concentration range of doses, choice of mammals, selection of demyelination disorders, markers associated with promotion of remyelination, formulation for administration, explained in the previous embodiments are applicable to this embodiment.

The preferred embodiments of the invention are further described in the following illustrative examples.

### EXAMPLES

Combination of Cuprizone and Rapamycin-Mediated

### Demyelination/Remyelination

The Eight-week-old male C56BL/6 J mice were selected and randomized into ten animals in each group (Table 1). Demyelination was induced by 0.3% cuprizone in combination with rapamycin (Hilary H S, Kathryn K B, Daniela C P, Priya N, Wendy B M. A New Model of Cuprizone-Mediated Demyelination/Remyelination. Am Society Neurochem. 2014: 1-16). Briefly, groups undergo demyelination by feeding on a diet containing 0.3% cuprizone mixed into Rodent Diet. These mice received additionally intra-peritoneal rapamycin injections 5 days a week with a vehicle solution.

After 8 weeks of treatment, all cuprizone feedings and all injections of rapamycin was discontinued. The animals were subjected to behavioral analysis and at end of study, brain samples and serum samples were collected for estimation of Molecular and protein biomarkers by ELISA and Immunohistochemistry of brain was assessed for Demyelination/Remyelination.

TABLE 1
Animal groups for study

| Groups | Number of Animals |
|---|---|
| Group I- Normal Control | 10 |
| Group II- Demyelinated Control | 10 |
| Group III- Withaferin A | 10 |
| Group IV- BDMC | 10 |
| Group V- Withaferin A + BDMC | 10 |

Dose Response Study

Brain-derived neurotrophic factor (BDNF) plays a role in both normal developmental myelination and in remyelination after some forms of demyelinating injury through regulation of proliferation and differentiation of oligodendrocyte proliferation cells (OPCs). A large body of neuropathological, experimental and clinical evidence shows that BDNF may play an important role in neuroinflammation modulation, neuroprotection and neurorepair. Myelin oligodendrocyte glycoprotein (MOG) is a glycoprotein important in the myelination of nerves in the central nervous system (CNS). In humans this protein is encoded by the MOG gene. It serves as a necessary “adhesion molecule” to provide structural integrity to the myelin sheath.

Withaferin A or BDMC were administered orally once per day to mouse, for 8 weeks after treatment with Cuprizone and Rapamycin (Table 1). The dose response of BDMC or Withaferin was assessed by measuring concentration of biomarkers, Myelin Oligodendrocyte Glycoprotein (MOG) (FIG. 1) Brain Derived Neurotrophic Factor (BDNF) (FIG. 2), in serum. This was used as a measure to find the optimum effective dose of Withaferin A (25 mg/kg) and BDMC (12.5 mg/kg) and effectively, the total concentration of 37.5 mg/kg (Withaferin A (25 mg/kg)+BDMC (12.5 mg/kg) was preferred for testing potencies for remyelination in assays. It must be noted that these concentrations are by no means a limitation rather embodiments cover the scope of concentration ranges.

Brain Tissue Preparation for Estimation of Molecular Protein Markers

Before homogenization for each assay (Magari, K., Miyata, S., Ohkubo, Y., Mutoh, S., 2004. Inflammatory cytokine levels in paw tissues during development of rat collagen-induced arthritis: effect of FK506, an inhibitor of T cell activation. Inflamm. Res. 53, 469-474), the brain of the animals was removed and rinsed with ice-cold isotonic saline and were added to 4 ml/g tissue of extraction buffer containing 1 mM phenyl methyl sulfonyl fluoride, 1 mg/ml aprotinin, and 0.05% Tween 20 in phosphate buffered saline. Tissues were homogenized on ice with a polytron, and homogenate was centrifuged at 5000 g for 15 min. Aliquots of the supernatant were separated and used for biochemical analysis. Supernatants were stored at −80° C. until cytokine analysis.

Estimation of Biomarkers in Brain Tissue Homogenate

The supernatant from brain tissue homogenate were separated and used for biomarker analysis. Myelin Oligodendrocyte Glycoprotein (FIG. 1), Platelet derived growth factor receptor alpha (FIG. 4), chondroitin sulfate proteoglycan 4 (FIG. 5), Myelin basic protein (FIG. 3), Transient receptor potential ankyrin 1 (FIG. 6), Amphoterin-induced protein 3 (FIG. 7), C-reactive protein (FIG. 8) and CXC chemokine receptor 2 (FIG. 9), were estimated using commercially available kits based on sandwich and competitive ELISA technique according to the manufacturers' instructions. All cytokine concentrations were carried out by means of colorimetric measurement at 450 nm on an ELISA plate reader by interpolation from a standard curve.

Proliferation of Glycoproteins:

Myelin oligodendrocyte glycoprotein (MOG) is a glycoprotein important in the myelination of nerves in the central nervous system (CNS). In humans this protein is encoded by the MOG gene. It serves as a necessary “adhesion molecule” to provide structural integrity to the myelin sheath. Differentiation of OPCs into pre-myelinating oligodendrocytes is associated with an increase in cellular complexity and the expression of myelin associated proteins and lipids, including myelin basic protein (MBP), myelin associated glycoprotein (MOG). During this stage, cells are highly susceptible to the induction of cell death, as 50% of the pre-myelinating oligodendrocytes undergo apoptotic cell death. If pre-myelinating oligodendrocytes each associate with an appropriate population of axons they mature into myelinating oligodendrocytes. Animal groups carrying Withaferin A (25 mg/kg) or BDMC (12.5 mg/kg) resulted in proliferation of Myelin basic protein (MBP) by 64 and 122% respectively. The animal group with combination (37.5 mg/kg) of Withaferin A and BDMC resulted in proliferation of Myelin basic protein (MBP) by 176%, compared to the demyelination control (FIG. 3), which is in the absence of Withaferin A or BDMC or the combination.

Upregulation of Growth Factors:

At the early stages of oligodendrocyte lineage commitment, oligodendrocyte precursor cell (OPCs) expresses a characteristic molecular phenotype that includes expression of the platelet derived growth factor receptor alpha(PDGFRa). OPCs are highly proliferative in response to growth factors PDGFa and fibroblast growth factor (FGF) and disperse widely throughout the developing CNS. Animal groups carrying Withaferin A (25 mg/kg) or BDMC (12.5 mg/kg) resulted in upregulation of PDGFRa by 22 and 73% respectively. The animal group with combination (37.5 mg/kg) of Withaferin A and BDMC resulted in upregulation of PDGFRa by 138%, compared to the demyelination control (FIG. 4). Similarly, during developmental stages oligodendrocyte precursor cells expressing the proteoglycan Chondroitin Sulfate Proteoglycan 4 (NG2) are expressed in the adult CNS, indicating remyelination in demyelinating diseases. NG2 was upregulated by 30 and 90% for Withaferin A (25 mg/kg) or BDMC (12.5 mg/kg) respectively. The combination (37.5 mg/kg) of Withaferin A and BDMC resulted in upregulation of PDGFRa by 159%, compared to the demyelination control (FIG. 5), which is in the absence of Withaferin A or BDMC or the combination.

Downregulation of Inflammatory Biomarkers:

Transient receptor potential ankyrin 1 (TRPA1) is a non-selective cation channel that plays a role in inflammatory pain sensation and nociception and a potential regulator in emotion, cognition and social behaviour. It is involved in the myelin Ca2+ accumulation process activating NADPH oxidase 1 (NOX1), which acts by releasing oxidant molecules and increasing their expression, to produce more ROS, eventually exacerbating oxidative stress. Inhibition of TRPA1 receptors might successfully diminish the degenerative pathology in multiple sclerosis and could be a promising therapeutic target to limit central nervous system damage in demyelinating diseases. Animal groups carrying withaferin A (25 mg/kg) or BDMC (12.5 mg/kg) resulted in down regulation of Transient receptor potential ankyrin 1 (TRPA1) inflammatory marker, by 43% and 17% respectively. The animal group with combination (37.5 mg/kg) of Withaferin A and BDMC resulted in down regulation of (TRPA1) inflammatory marker, by 46% compared to the demyelination control (FIG. 6), which is in the absence of Withaferin A or BDMC or the combination.

AMIGO3 exerts inhibitory effect on the axonal growth of central nervous system neurons. The increase in spinal cord AMIGO3 expression in MS suggests that this protein plays a role in the acute stages of oligodendrocyte injury. In this context, therapies targeted against AMIGO3 provides benefit in terms of promoting OPC differentiation and remyelination in the early stages of demyelinating diseases (Neural Regeneration Research 2017 August; 12(8): 1247-1251). Animal groups carrying Withaferin A (25 mg/kg) or BDMC (12.5 mg/kg) resulted in down regulation of Amphoterin-induced protein 3 (AMIGO3) inflammatory marker by 36% and 27% respectively. The animal group with combination (37.5 mg/kg) of Withaferin A and BDMC resulted in down regulation of AMIGO3 by 43%, compared to the demyelination control (FIG. 7), which is in the absence of Withaferin A or BDMC or the combination.

The serum components which are acute phase reactants that become elevated during inflammation and tissue destruction, C-reactive protein was measured in patients with multiple sclerosis (MS) hospitalized with acute exacerbations. Significantly elevated levels of CRP was found in 12 of 13 patients with clinically active MS. Serial studies in two patients revealed that clinical improvement was accompanied by a decline in the serum levels of CRP. These findings suggest that measurement of these serum proteins may be of value in assessing progress of disease activity in MS patients (P C Dowling, S D Cook, Disease markers in acute multiple sclerosis, 1976 October; 33(10):668-7). Animal groups carrying Withaferin A (25 mg/kg) or BDMC (12.5 mg/kg) resulted in down regulation of C-reactive Protein (CRP) inflammatory marker by 32 and 35% respectively. The animal group with combination (37.5 mg/kg) of Withaferin A and BDMC resulted in down regulation of CRP inflammatory marker by 61%, compared to the demyelination control (FIG. 8), which is in the absence of Withaferin A or BDMC or the combination.

CXCR2 is the inflammation-inducible receptor of CXCL 5 and CXCL 8 expressed by human brain endothelium. Chemokines ligand (CXCL) 5 and 8 are overexpressed in patients with multiple sclerosis. CXCR2 is weakly expressed in resting brain endothelium but highly inducible by inflammatory stimuli. CXCR2 is closely involved in the chemotaxis of neutrophils to sites of injury through high-affinity binding with a gradient of its cognate ligands. Indeed, several preclinical mouse models of MS have shown that CXCR2-mediated recruitment of neutrophils influences the severity of demyelination (Carlson et al., 2008; Simmons et al., 2014; Liu el al., 2015a; Marro el al., 2016, Gris el al., 2018). Animal groups carrying Withaferin A (25 mg/kg) or BDMC (12.5 mg/kg) resulted in down regulation of CXC chemokine receptor 2 (CXCR2) inflammatory marker by 32 and 35% respectively. The animal group with combination (37.5 mg/kg) of Withaferin A and BDMC resulted in down regulation of CXC chemokine receptor 2 (CXCR2) inflammatory marker by at least 50%, or preferably by at least 55%, or most preferably by 65%, compared to the demyelination control (FIG. 9), which is in the absence of Withaferin A or BDMC or the combination.

Estimation of Biomarkers in Serum:

Blood was collected from the retro-orbital plexus of the experimental animals, no anti-coagulant was added to the blood, and it was made to stand at room temperature for 1 h. The blood was then centrifuged, and clear serum was separated and stored for analysis.

Samples from different groups of animals were prepared for the analysis of cytokines and mediators at the termination of the experiment. Brain Derived Neurotrophic Factor (BDNF), C-Reactive Protein were estimated using commercially available kits based on sandwich and competitive ELISA technique according to the manufacturers' instructions. All cytokine concentrations were carried out by means of colorimetric measurement at 450 nm on an ELISA plate reader by interpolation from a standard curve

Motor Coordination and Learning: Rotarod Test

Motor coordination and balance were evaluated in a Rotarod apparatus which consists of a motor-driven rotating rod whose speed can be adjusted. All mice were evaluated on the Rotarod three times a day for two consecutive days, with the rotation set at 15 to 16 revolutions per minute (rpm) (Rozas, G.; Guerra, M. J.; Labandeira Garcia, J. L. An automated Rotarod method for quantitative drug-free evaluation of overall motor deficits in rat models of Parkinsonism. Brain Res. Protoc. 1997, 2, 75-84). To test the performance, the mice were placed on the rotating cylinder at an angle of 45 with an initial rotation speed of 16 rpm, and were allowed to run for 60 s. The trial was repeated after 5-10 min. The falls and flips (when the animal hangs on to the cylinder and continues all the way around) were recorded within 60 s of each trial. Animal groups carrying Withaferin A (25 mg/kg) or BDMC (12.5 mg/kg) resulted in improved motor co-ordination in 5 out of 10 mammals, whereas the animal group with combination (37.5 mg/kg) of Withaferin A and BDMC resulted in improved motor co-ordination in 7 out of 10 mammals, compared to 3 out of 10 in demyelinated control mammals (Table 2).

TABLE 2
Results from Rotarod Test

| | No. of mice which stayed on |
|---|---|
| Treatment | rota rod/total no. of animals |
| Normal Control | 8/10 |
| Demyelinated Control | 3/10 |
| Withaferin 25 mg/kg | 5/10 |
| BDMC 12.5 mg/kg | 5/10 |
| Withaferin + BDMC 25 + 12.5 mg/kg | 7/10 |

Grip Strength Test:

The grip strength test allows for the assessment of neuromuscular functions by determining the maximal peak force developed by a rodent when the operator tries to pull it out of a specially designed grid, available for both the fore and hind limbs. In this context, changes in grip strength peak values of the rodents are interpreted as evidence of motor neurotoxicity. Forelimb strength (g force) was measured with a grip strength tester to detect contralateral paw weakness (Seung Ro Han et al. Differential Expression of miRNAs and Behavioral Change in the Cuprizone-Induced Demyelination Mouse Model. International Journal of Molecular Sciences. 2020, 21, 646). After both forelimbs of the mouse were loosened by pulling the tail, the maximal force was recorded. Each mouse was subjected to three trials of each test, and the mean values (g) were calculated. Animal groups carrying Withaferin A (25 mg/kg) or BDMC (12.5 mg/kg) and animal group with the combination (37.5 mg/kg) of two showed 25%, 37.5%, and 58% improvement in the grip strength respectively compared to the demyelinated control (FIG. 10), which is in the absence of Withaferin A or BDMC or the combination.

Locomotor Activity Test:

Actophotometer (Activity cage) is designed to study the spontaneous or induced or locomotor activity in small animals like mice or rats. In this apparatus, the Optical sensors and emitters are present to record the horizontal movements of the animals on a six-digit digital counter display. Each animal was placed in Actophotometer for 5 minutes and basal activity score was recorded for all animals. Each animal was treated with respective drugs and activity score was recorded after 1, 2, and 3 hours. Animal group carrying Withaferin A (25 mg/kg) showed 30 to 40% improvement compared to the demyelinated control, and the animal group with BDMC (12.5 mg/kg) showed 25 to 30% improvement, and the combination (37.5 mg/kg) showed 60 to 65% improvement compared to the demyelinated control (Table 3), which is in the absence of Withaferin A or BDMC or the combination.

TABLE 3
Results from Locomotor Activity Test,

| Doses | 1 hr after | 2 hr after | 3 hr after |
|---|---|---|---|
| (mg/kg p.o.) | treatment | treatment | treatment |
| Normal Control | 342 ± 22.66 | 330 ± 17.22 | 310 ± 10.12 |
| Demyelinated | 170 ± 20.18 | 158 ± 14.02 | 148 ± 14.48 |
| Control | | | |
| Withaferin A | 240 ± 17.22** | 210 ± 17.23* | 200 ± 9.01* |
| 25 mg/kg | | | |
| BDMC 12.5 mg/kg | 220 ± 21.11* | 200 ± 10.70* | 190 ± 7.35* |
| Withaferin A + | 275 ± 9.12** | 260 ± 8.56** | 245 ± 2.35** |
| BDMC 37.5 mg/kg | | | |

Values are expressed as Mean ± S.E.;
Number of animals in each group: 10;
p value: *<0.01;
**<0.001

Immunohistochemistry

For visualization of mature neurons, sections were boiled in citrate buffer solution (10 mM, pH 6.0) for antigen retrieval, followed by 1 h blocking in PBS solution containing 10% goat serum and 0.3% Triton and incubated overnight with glutathione S-transferase Pi (GST-π) antibody (Enzo Life Sciences, Farmingdale, N.Y.) at 4° C. Sections were then rinsed in PBS for washing and further incubated for 3 h in fluorescent secondary antibody (Alexa Fluor Goat anti Rabbit 543, ThermoFisher, 1:200) and to-pro-3 (1:1000, Thermo Fisher, T3605) for nuclear stain (FIG. 7)(Qili Yu, Ryan Hui, Jiyoung Park, Yangyang Huang, Alexander W. Kusnecov et al. Strain differences in cuprizone induced demyelination. Cell Biosci (2017) 7:59). Normal control showed architecture of neurons with intact myelin fibers. The cortex of cerebrum showed no remarkable changes with respect to neurons in the normal control (FIG. 11), whereas the demyelinated control (FIG. 12) showed degenerated Purkinje cells with pyknotic cell bodies and inconspicuous dendritic processes with multifocal vacuolations, demyelination and focal purkinje cell degeneration. In contrast, Withaferin A (25 mg/kg, FIG. 13) showed the histoarchitectural presentation of the cerebellar cortical layer, which showed few degenerated Purkinje cells with pyknotic cell bodies and inconspicuous dendritic processes, whereas BDMC (12.5 mg/kg, FIG. 14) showed Purkinje cells with the Purkinje layer to be better stained with fewer degenerative properties when compared to the untreated animals, the given tissue also showed multifocal neuronal degeneration with moderate demyelination. The combination of Withaferin A+BDMC (37.5 mg/kg, FIG. 15) showed significantly lesser degenerated Purkinje cells and the granular layer was less dense than that of the demyelinated control slides and also showed for remyelination in certain areas and proliferation and granular cell layer.

Formulations Containing Withaferin a and BDMC

The composition is formulated along with pharmaceutically/nutraceutically acceptable excipients, adjuvants, diluents, stabilizing agents, dispersible gums, bioavailability enhancers or carriers and administered orally in the form of tablets, capsules, syrups, gummies, powders, suspensions, emulsions, chewables, candies or eatables.

In a related aspect the bioavailability enhancer is selected from the group of piperine (BioPerine®), quercetin, garlic extract, ginger extract, and naringin. In another related aspect, the stabilizing agent is selected from the group consisting rosmarinic acid, butylated hydroxyanisole, butylated hydroxytoluene, sodium metabisulfite, propyl gallate, cysteine, ascorbic acid and tocopherols. In yet another related aspect, the dispersible gums are selected from the group consisting of Agar, Alginate, Carrageenan, Gum Arabic, Guar Gum, Locust Bean Gum, Konjac Gum, Xanthan Gum and Pectin.

Tables 4-8 provide illustrative examples of nutraceutical formulations containing bisdemethoxycurcumin

TABLE 4
Tablet

Active Ingredients
Withaferin A
Bisdemethoxycurcumin (BDMC)
Excipients
Microcrystalline cellulose, Colloidal silicon dioxide, Magnesium
stearate, BioPerine ®, Polyvinylpyrrolidone/starch/Hydroxy propyl
methyl cellulose, Hydroxy propyl cellulose

TABLE 5
Capsule

Active Ingredients
Withaferin A
Bisdemethoxycurcumin (BDMC)
Excipients
Microcrystalline cellulose, BioPerine ®

TABLE 6
Powder

Active Ingredients
Withaferin A
Bisdemethoxycurcumin (BDMC)
Excipients
BioPerine ®,

TABLE 7
Gummy formulation

Active Ingredients
Withaferin A
Bisdemethoxycurcumin (BDMC)
Excipients
BioPerine ®, Gelatin (270 Bloom Mesh 10), Refined Sugar, Glucose
Corn Syrup, Citric Acid, Lactic Acid, Water, Natural Mango Flavor
M38630, Tartaric Acid, Refined Sugar

TABLE 8
Candy formulation

Active Ingredients
Withaferin A
Bisdemethoxycurcumin (BDMC)
Excipients
BioPerine ®, Sucrose, Liquid Glucose, Flavoring agent, Menthol,
Acidulants (Citric acid/Tartaric Acid/Maleic Acid), Purified water

The above formulations are merely illustrative examples, any formulation containing the above active ingredient intended for the said purpose will be considered equivalent. Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

We claim:

1. A composition comprising Withaferin A and Bisdemethoxycurcumin (BDMC).

2. The composition as claimed in claim 1, wherein Withaferin A and BDMC are in 3:1 w/w: 1-3 w/w.

3. The composition as claimed in claim 1, the composition further comprises stabilizing agents, bioavailability enhancers and antioxidants, pharmaceutically or nutraceutically or cosmeceutically accepted excipients and enhancers, administered orally in the form of tablets, capsules, syrups, gummies, powders, suspensions, emulsions, chewables, candies or eatables.

4. A method for therapeutic management of a demyelinating disorder in a mammal, said method comprising steps of:
(a) identifying a mammal showing symptoms of demyelination; and
(b) administering to the mammal, an effective dose of a composition comprising Bisdemethoxycurcumin (BDMC), to bring about the effect of promoting remyelination.

5. The method as in claim 4, wherein the composition further comprises withaferin A.

6. The method as in claim 4, wherein the effective dose of BDMC and withaferin A is individually selected from 3 mg/kg to 50 mg/kg bodyweight individually of the said mammal.

7. The method as in claim 4, wherein the total effective dose of the composition is selected from 35 to 40 mg/kg bodyweight, wherein the dose of Withaferin A is 25 mg/kg and BDMC is 12.5 mg/kg.

8. The method as in claim 4, wherein the demyelinating disorder is selected from the group consisting of Multiple Sclerosis, Acute Disseminated Encephalomyelitis (ADEM), Balo's Disease (Concentric Sclerosis), Charcot-Marie-Tooth Disease (CMT), Guillain-Barre Syndrome (GBS), HTLV-I Associated Myelopathy (HAM), Neuromyelitis Optica (Devic's Disease), Schilder's disease, and Transverse Myelitis.

9. The method as in claim 4, wherein management of the demyelinating disorder in the mammal is brought about by promoting remyelination, by increasing proliferation of glycoproteins involved in myelin formation, upregulation of growth factors, down regulation of inflammatory markers and increasing neuromuscular coordination.

10. The method as in claim 9, wherein the glycoproteins are selected from the group consisting of Myelin basic protein (MBP) and Myelin Oligodendrocyte Glycoprotein (MOG).

11. The method as in claim 9, wherein the growth factors are selected from the group consisting of Platelet Derived Growth Factor Receptor alpha (PDGFRa) and Chondroitin Sulfate Proteoglycan 4 (NG2).

12. The method as in claim 9, wherein the inflammatory markers are selected from the group consisting of Transient receptor potential ankyrin 1 (TRPA1), Amphoterin-induced protein 3 (AMIGO3), C-reactive Protein (CRP), and CXC chemokine receptor 2 (CXCR2).

13. The method as in claim 9, wherein the neuro-muscular coordination is selected from the group consisting of grip strength, motor coordination, learning and locomotor activity.

14. The method as in claim 4, wherein the composition further comprises stabilizing agents, bioavailability enhancers and antioxidants, pharmaceutically or nutraceutically or cosmeceutically accepted excipients and enhancers and administered orally in the form of tablets, capsules, syrups, gummies, powders, suspensions, emulsions, chewables, candies or eatables.

15. A method of promoting remyelination of a mammalian neuron, said method comprising: bringing into contact an effective dose of a composition comprising Bisdemethoxycurcumin (BDMC) to the said mammalian neuron, showing characteristic of demyelination, to bring about the effect of promoting remyelination.

16. The method as in claim 15, wherein the neuron is selected from the group consisting of central and peripheral nervous system.

17. The method as in claim 15, wherein the composition further comprises withaferin A.

18. The method as in claim 15, wherein the effective dose of BDMC and withaferin A is individually selected from 3 mg/kg to 50 mg/kg individually of the said neuron.

19. The method as in claim 15, wherein the total effective dose of the composition is selected from 35 to 40 mg/kg of the said neuron, wherein the dose of Withaferin A is 25 mg/kg and BDMC is 12.5 mg/kg.

20. The method as in claim 15, wherein promoting remyelination is effective in management of demyelinating disorders are selected from the group consisting of Multiple Sclerosis, Acute Disseminated Encephalomyelitis (ADEM), Balo's Disease (Concentric Sclerosis), Charcot-Marie-Tooth Disease (CMT), Guillain-Barre Syndrome (GBS), HTLV-I Associated Myelopathy (HAM), Neuromyelitis Optica (Devic's Disease), Schilder's disease, and Transverse Myelitis.

21. The method as in claim 15, wherein remyelination is promoted by increasing proliferation of glycoproteins involved in myelin formation, upregulation of growth factors, down regulation of inflammatory markers and increasing neuromuscular coordination.

22. The method as in claim 21, wherein the glycoproteins are selected from the group consisting of Myelin basic protein (MBP) and Myelin Oligodendrocyte Glycoprotein (MOG).

23. The method as in claim 21, wherein the growth factors are selected from the group consisting of Platelet Derived Growth Factor Receptor alpha (PDGFRa) and Chondroitin Sulfate Proteoglycan 4 (NG2).

24. The method as in claim 21, wherein the inflammatory markers are selected from the group consisting of Transient receptor potential ankyrin 1 (TRPA1), Amphoterin-induced protein 3 (AMIGO3), C-reactive Protein (CRP), and CXC chemokine receptor 2 (CXCR2).

25. The method as in claim 21, wherein the neuro-muscular coordination are selected from the group consisting of grip strength, motor coordination, learning and locomotor activity.

26. The method as in claim 15, wherein the composition further comprises stabilizing agents, bioavailability enhancers and antioxidants, pharmaceutically or nutraceutically or cosmeceutically accepted excipients and enhancers and administered orally in the form of tablets, capsules, syrups, gummies, powders, suspensions, emulsions, chewables, candies or eatables.
